(19) United States
(12) Patent Application Publication
(10) Pub. No.: US 2012/0053391 A1
(43) Pub. Date: Mar. 1, 2012

(54) SHAPED AND STEERED ULTRASOUND FOR DEEP-BRAIN NEUROMODULATION

(76) Inventor: David J. Mishelevich, Playa del Rey, CA (US)

(21) Appl. No.: 13/200,903

(22) Filed: Jan. 15, 2011

Related U.S. Application Data
(60) Provisional application No. 61/295,759, filed on Jan. 18, 2010.

(51) Int. Cl. A61N 2/00; A61N 7/00

(52) U.S. Cl. 600/9; 601/2

(57) ABSTRACT

Disclosed are devices for producing shaped or steered ultrasound for non-invasive deep brain or superficial neuromodulation impacting one or a plurality of points in a neural circuit. Depending on the application this can produce short-term effects (as in the treatment of post-surgical pain) or long-term effects in terms of Long-Term Potentiation (LTP) or Long-Term Depression (LTD) to treat indications such as neurologic and psychiatric conditions. The ultrasound transducers are used with control of direction of the energy emission, control of intensity, control of frequency for up-regulation or down-regulation, and control of phase/intensity relationships for focusing on neural targets.

### CROSS REFERENCE TO RELATED APPLICATIONS

This patent application claims priority to provisional patent Application No. 61/295,759, filed Jan. 18, 2010, entitled “SHAPED AND STEERED ULTRASOUND FOR DEEP-BRAIN NEUROMODULATION.” The disclosures of this patent application are herein incorporated by reference in their entirety.

### INCORPORATION BY REFERENCE

All publications, including patents and patent applications, mentioned in this specification are herein incorporated by reference in their entirety to the same extent as if each individual publication was specifically and individually indicated to be incorporated by reference.

### FIELD OF THE INVENTION

Described herein are systems and methods for Ultrasound Stimulation including one or a plurality of ultrasound sources for stimulation of target deep brain regions to up-regulate or down-regulated neural activity.

### BACKGROUND OF THE INVENTION

It has been demonstrated that focused ultrasound directed at neural structures can stimulate those structures. If neural activity is increased or excited, the neural structure is said to be up-regulated; if neural activated is decreased or inhibited, the neural structure is said to be down-regulated. Down regulation means that the firing rate of the neural target has its firing rate decreased and thus is inhibited and up regulation means that the firing rate of the neural target has its firing rate increased and thus is excited. Neural structures are usually assembled in circuits. For example, nuclei and tracts connecting them make up a circuit. The potential application of ultrasonic therapy of deep-brain structures has been suggested previously (Gavrilov L R, Tsirulnikov E M, and I A Davies, “Application of focused ultrasound for the stimulation of neural structures,” Ultrasound Med Biol. 1996; 22(2):179-92. and S. J. Norton, “Can ultrasound be used to stimulate nerve tissue?,” BioMedical Engineering OnLine 2003, 2:6). Norton notes that while Transcranial Magnetic Stimulation (TMS) can be applied within the head with greater intensity, the gradients developed with ultrasound are comparable to those with TMS. It was also noted that monophasic ultrasound pulses are more effective than biphasic ones. Instead of using ultrasonic stimulation alone, Norton applied a strong DC magnetic field as well and describes the mechanism as that given that the tissue to be stimulated is conductive that particle motion induced by an ultrasonic wave will induce an electric current density generated by Lorentz forces.

The effect of ultrasound is at least two fold. First, increasing temperature will increase neural activity. An increase up to 42 degrees C. (say in the range of 39 to 42 degrees C.) locally for short time periods will increase neural activity in a way that one can do so repeatedly and be safe. One needs to make sure that the temperature does not rise about 50 degrees C. or tissue will be destroyed (e.g., 56 degrees C. for one second). This is the objective of another use of therapeutic application of ultrasound, ablation, to permanently destroy tissue (e.g., for the treatment of cancer). An example is the ExAblate device from InSightec in Haifa, Israel. The second mechanism is mechanical perturbation. An explanation for this has been provided by Tyler et al. from Arizona State University (Tyler, W. J., Y. Tufail, M. Finsterwald, M. L. Tauchmann, E. J. Olsen, C. Majestic, “Remote excitation of neuronal circuits using low-intensity, low-frequency ultrasound,” PLoS One 3(10): e3511, doi:10.137/1/journal.pone.0003511, 2008)) where voltage gating of sodium channels in neural membranes was demonstrated. Pulsed ultrasound was found to cause mechanical opening of the sodium channels which resulted in the generation of action potentials. Their stimulation is described at Low Intensity Low Frequency Ultrasound (LILFU). They used bursts of ultrasound at frequencies between 0.44 and 0.67 MHz, lower than the frequencies used in imaging. Their device delivered 23 milliwatts per square centimeter of brain—a fraction of the roughly 180 mW/cm² upper limit established by the U.S. Food and Drug Administration (FDA) for womb-scanning sonograms; thus such devices should be safe to use on patients. Ultrasound impact to open calcium channels has also been suggested.

Alternative mechanisms for the effects of ultrasound may be discovered as well. In fact, multiple mechanisms may come into play, but, in any case, this would not effect this invention.

Approaches to date of delivering focused ultrasound vary. Bystritsky (U.S. Pat. No. 7,283,861, Oct. 16, 2007) provides for focused ultrasound pulses (FUP) produced by multiple ultrasound transducers (said preferably to number in the range of 300 to 1000) arranged in a cap place over the skull to effect a multi-beam output. These transducers are coordinated by a computer and used in conjunction with an imaging system, preferable an fMRI (functional Magnetic Resonance Imaging), but possibly a PET (Positron Emission Tomography) or V-EEG (Video-Electroencephalography) device. The user interacts with the computer to direct the FUP to the desired point in the brain, sees where the stimulation actually occurred by viewing the imaging result, and thus adjusts the position of the FUP according. The position of focus is obtained by adjusting the phases and amplitudes of the ultrasound transducers (Clement and Hynynen, “A non-invasive method for focusing ultrasound through the human skull,” Phys. Med. Biol. 47 (2002) 1219-1236). The imaging also illustrates the functional connectivity of the target and surrounding neural structures. The focus is described as two or more centimeters deep and 0.5 to 1000 mm in diameter or preferably in the range of 2-12 cm deep and 0.5-2 mm in diameter. Either a single FUP or multiple FUPs are described as being able to be applied to either one or multiple live neuronal circuits. It is noted that differences in FUP phase, frequency, and amplitude produce different neural effects. Low frequencies (defined as below 300 Hz.) are inhibitory. High frequencies (defined as being in the range of 500 Hz to 5 MHz are excitatory and activate neural circuits. This works whether the target is gray or white matter. Repeated sessions result in long-term effects. The cap and transducers to be employed are preferably made of non-ferrous material to reduce image distortion in fMRI imaging. It was noted that if after treatment the reactivity as judged with fMRI of the patient with a given condition becomes more like that of a normal patient, this may be indicative of treatment effectiveness. The FUP is to be applied 1 ms to 1 s before or after the imaging. In addition a CT (Computed Tomography) scan can be run to gauge the bone density and structure of the skull.

An alternative approach is described by Deisseroth and Schneider (U.S. patent application Ser. No. 12/263,026 published as US 2009/0112133 A1, Apr. 30, 2009) in which modification of neural transmission patterns between neural structures and/or regions is described using sound (including use of a curved transducer and a lens) or RF. The impact of Long-Term Potentiation (LTP) and Long-Term Depression (LTD) for durable effects is emphasized. It is noted that sound produces stimulation by both thermal and mechanical impacts. The use of ionizing radiation also appears in the claims.

Adequate penetration of ultrasound through the skull has been demonstrated (Hynynen, K. and FA Jolesz, “Demonstration of potential noninvasive ultrasound brain therapy through an intact skull,” Ultrasound Med Biol, 1998 Feb.; 24(2):275-83 and Clement G T, Hynynen K (2002) A non-invasive method for focusing ultrasound through the human skull. Phys Med Biol 47: 1219-1236.). Ultrasound can be focused to 0.5 to 2 mm as TMS to 1 cm at best.

### SUMMARY OF THE INVENTION

It is the purpose of this invention to provide a device for producing shaped or steered ultrasound for non-invasive deep brain or superficial stimulation impacting one or a plurality of points in a neural circuit to produce acute effects or Long-Term Potentiation (LTP) or Long-Term Depression (LTD) using up-regulation or down-regulation.

### BRIEF DESCRIPTION OF THE DRAWINGS

FIG. 1: Ultrasound transducer array configured to produce an elongated pencil-shaped focused field.

FIG. 2: Elongated ultrasound transducer array with sound conduction medium.

FIG. 3: Neural-circuit diagram for addiction.

FIG. 4: Physical target layout for addiction.

FIG. 5: Two ultrasound transducer arrays with different radii.

FIG. 6: Flat transducer array with interchangeable lenses.

FIG. 7: Linear ultrasound phased array with steered-beam linearly moving field.

FIG. 8: Combination of ultrasound transducer with TMS Coil.

### DETAILED DESCRIPTION OF THE INVENTION

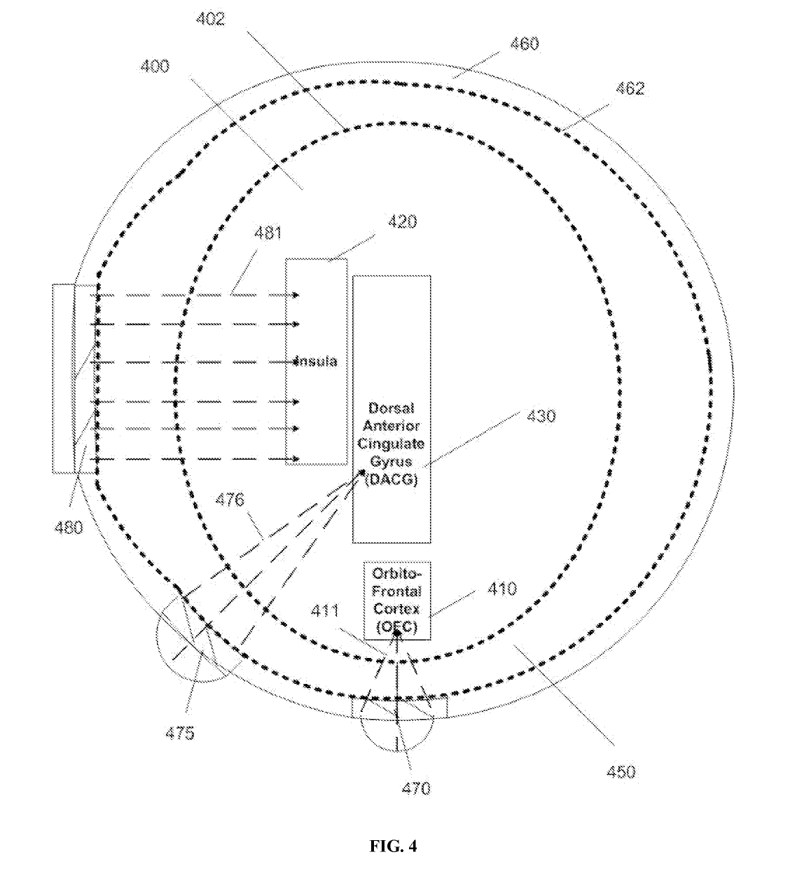

It is the purpose of this invention to provide a device for producing shaped or steered ultrasound for non-invasive deep brain or superficial stimulation impacting one or multiple points in a neural circuit to produce acute effects or Long-Term Potentiation (LTP) or Long-Term Depression (LTD) using up-regulation or down-regulation. For example, FIG. 3 illustrates the neural circuit for addiction.

The stimulation frequency for inhibition is 300 Hz or lower (depending on condition and patient). The stimulation frequency for excitation is in the range of 500 Hz to 5 MHz. In this invention, the ultrasound acoustic frequency is in range of 0.3 MHz to 0.8 MHz to permit effective transmission through the skull with power generally applied less than 180 mW/cm² but also at higher target- or patient-specific levels at which no tissue damage is caused. The acoustic frequency (e.g., 0.44 MHz that permits the ultrasound to effectively penetrate through skull and into the brain) is gated at the lower rate to impact the neuronal structures as desired (e.g., say 300 Hz for inhibition (down-regulation) or 1 kHz for excitation (up-regulation). If there is a reciprocal relationship between two neural structures (i.e., if the firing rate of one goes up the firing rate of the other will decrease), it is possible that it would be appropriate to hit the target that is easiest to obtain the desired result. For example, one of the targets may have critical structures close to it so if it is a target that would be down regulated to achieve the desired effect, it may be preferable to up-regulate its reciprocal more-easily-accessed or safer reciprocal target instead. The frequency range allows penetration through the skull balanced with good neural-tissue absorption. Ultrasound therapy can be combined with therapy using other devices (e.g., Transcranial Magnetic Stimulation (TMS), transcranial Direct Current Stimulation (tDCS), and/or Deep Brain Stimulation (DBS) using implanted electrodes).

The lower bound of the size of the spot at the point of focus will depend on the ultrasonic frequency, the higher the frequency, the smaller the spot. Ultrasound-based neuromodulation operates preferentially at low frequencies relative to say imaging applications so there is less resolution. As an example, let us have a hemispheric transducer with a diameter of 3.8 cm. At a depth approximately 7 cm the size of the focused spot will be approximately 4 mm at 500 kHz where at 1 Mhz, the value would be 2 mm. Thus in the range of 0.4 MHz to 0.7 MHz, for this transducer, the spot sizes will be on the order of 5 mm at the low frequency and 2.8 mm at the high frequency.

Transducer array assemblies of the type used in this invention may be supplied to custom specifications by Imasonic in France (e.g., large 2D High Intensity Focused Ultrasound (HIFU) hemispheric array transducer)(Fleury G., Berriet, R., Le Baron, O., and B. Huguenin, “New piezocomposite transducers for therapeutic ultrasound,” 2^{nd} International Symposium on Therapeutic Ultrasound—Seattle—31/07—Feb. 8, 2002), typically with numbers of sound transducers of 300 or more. Keramos-Etalon and Blatek in the U.S. are other custom-transducer suppliers. The power applied will determine whether the ultrasound is high intensity or low intensity (or medium intensity) and because the sound transducers are custom, any mechanical or electrical changes can be made, if and as required.

The locations and orientations of the transducers in this invention can be calculated by locating the applicable targets relative to atlases of brain structure such as the Tailarach atlas or established though fMRI, PET, or other imaging of the head of a specific patient. Using multiple ultrasound transducers two or more targets can be targeted simultaneously or sequentially. Using a phased array with ability to focus and steer the beam, two or more targets can be targeted sequentially. The ultrasonic firing patterns can be tailored to the response type of a target or the various targets hit within a given neural circuit.

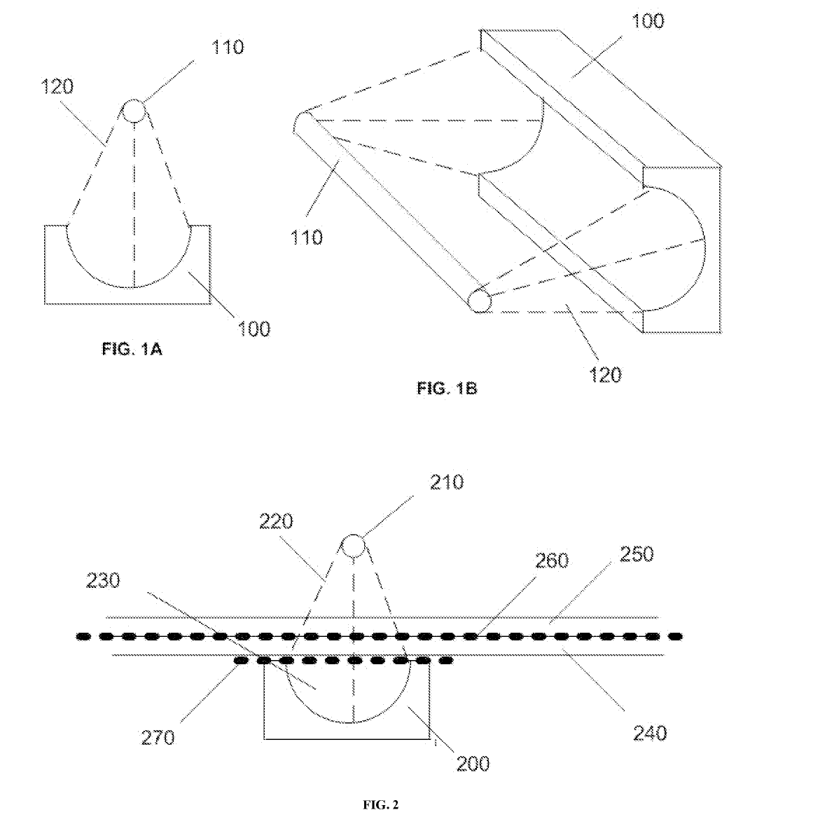

FIG. 1 shows an ultrasound transducer array configured to produce an elongated pencil-shaped focused field. Such an array would he applied to stimulate an elongated target such as the Dorsal Anterior Cingulate Gyms (DACG) or the Insula. Note that one embodiment is a swept-beam transducer with the capability of sweeping the sound field over any portion of the length of the ultrasound transducer. Thus it is possible to determine over what length of a target that the ultrasound is applied. For example, one could apply ultrasound to only the anterior portion of the target. Also, by rotating or tilting a transducer in a holder, one can vertically target such as aiming the sound field at the superior portion of a target. In FIG. 1A, an end view of the array is shown with curved-cross section ultrasonic array 100 forming a sound field 120 focused on target 110. FIG. 1B shows the same array in a side view, again with ultrasound array 100, target 110, and focused field 120.

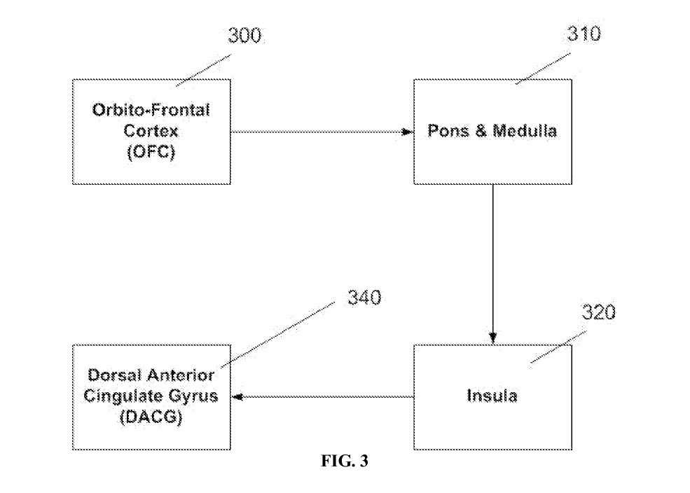

FIG. 2 illustrates the elongated ultrasound transducer array shown in FIG. 1 (now with ultrasound-transducer array 200, target 210, and focused ultrasound field 220), but in this case showing head layer 250 and sound-conduction medium 230 in place. Ultrasound is transmitted through fitted sound-conduction medium 230, a layer of conduction gel 270 providing the interface to solid sound-conduction medium 240, and a layer of conduction gel 260 providing interface to the head layer. Examples of sound-conduction media are Dermasol from California Medical Innovations or silicone oil in a containment pouch.

An example of a neural circuit for addiction is shown in FIG. 3. In this circuit, the elements are Orbito-Frontal Cortex (OFC) 300, Pons & Medulla 310, Insula 320, and Dorsal Anterior Cingulate Gyms (DACG) 340. One or more targets can be targeted simultaneously or sequentially. Down regulation means that the firing rate of the neural target has its firing rate decreased and thus is inhibited and up regulation means that the firing rate of the neural target has its firing rate increased and thus is excited. For the treatment of addiction, the OFC 300, Insula 320, and DACG 340 would all be down regulated. The ultrasonic firing patterns can be tailored to the response type of a target or the various targets hit within a given neural circuit.

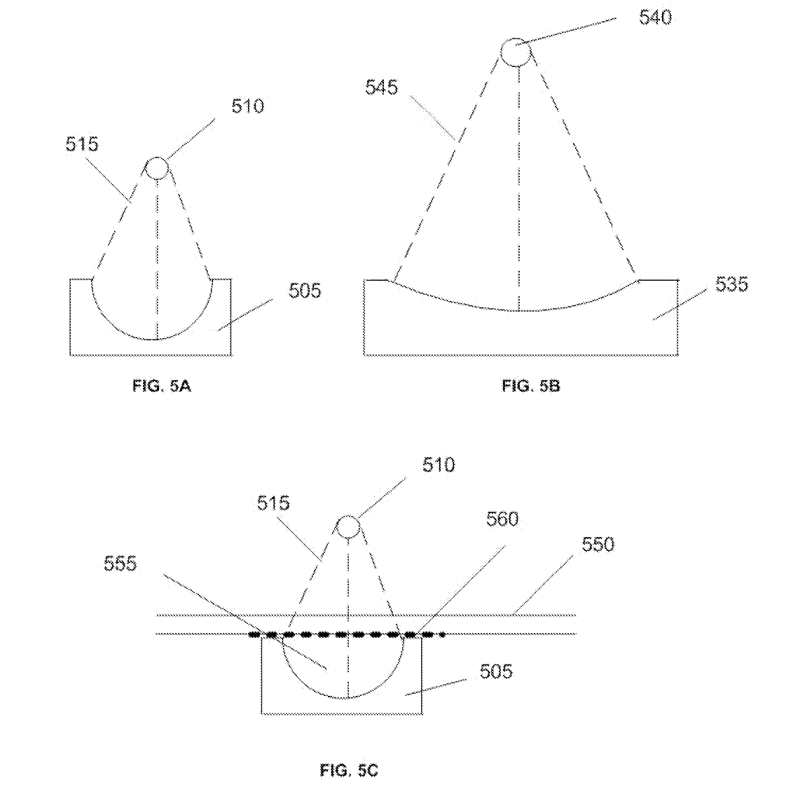

In FIG. 4, the physical target layout for addiction for the targets shown in FIG. 3 has within head 400 targets Orbito-Frontal Cortex (OFC) 410, Dorsal Anterior Cingulate Gyms (DACG) 430, and Insula 420. Sound field 411 emanating from ultrasound transducer 470 is focused on Orbito-Frontal Cortex (OFC) 410. Sound field 476 emanating from ultrasound transducer 475 is focused on Dorsal Anterior Cingulate Gyms (DACG) 430. Sound field 481 emanating from ultrasound transducer 480 is focused on Insula 420. All of the ultrasound transducers are mounted on frame 460 with the ultrasound conducted through conductive gel layer 462, conductive medium 450, and conductive gel layer 402 that provides the interface to head 400.

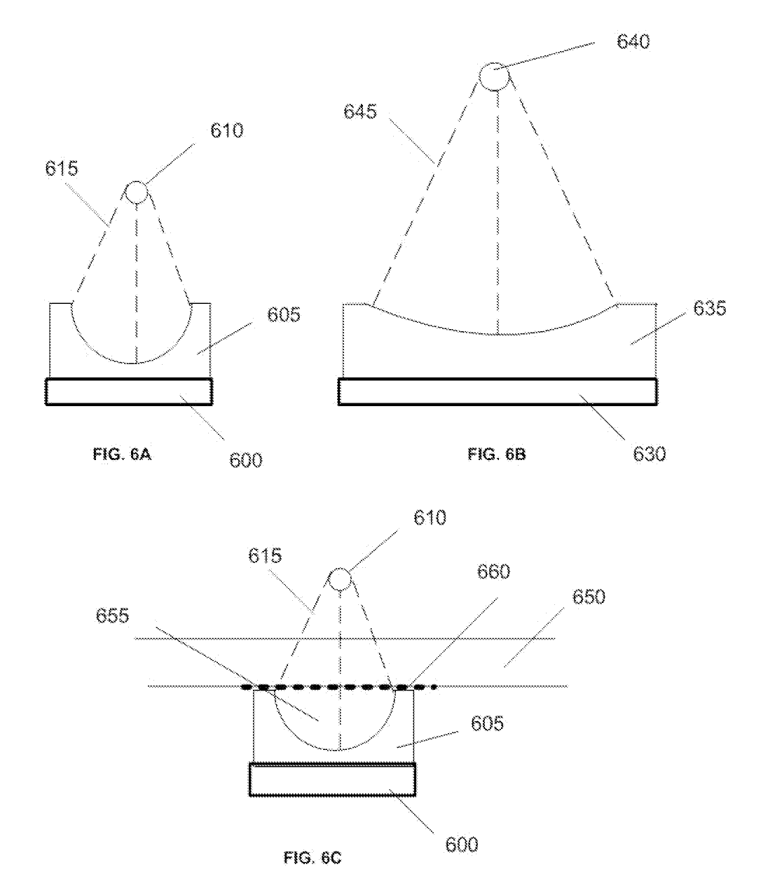

FIG. 5 demonstrates two ultrasound transducer arrays with different radii. The array with the shorter focal length in FIG. 5A has transducer array 505 focusing sound field 505 at target 510. In FIG. 5B, the array with the longer focal length because of the larger radius has transducer array 535 focusing sound field 545 at target 540. In order to work, there must be a medium between the transducer array and the head to conduct the sound. In FIG. 5C shows the transducer array 505 of FIG. 5A with sound field 515 focused on target 510 with sound conduction media in place between array 505 and head 550. The conduction mechanism consists of hemispheric conduction medium 555 and conducting-gel layer 560 providing the physical interface to head 550.

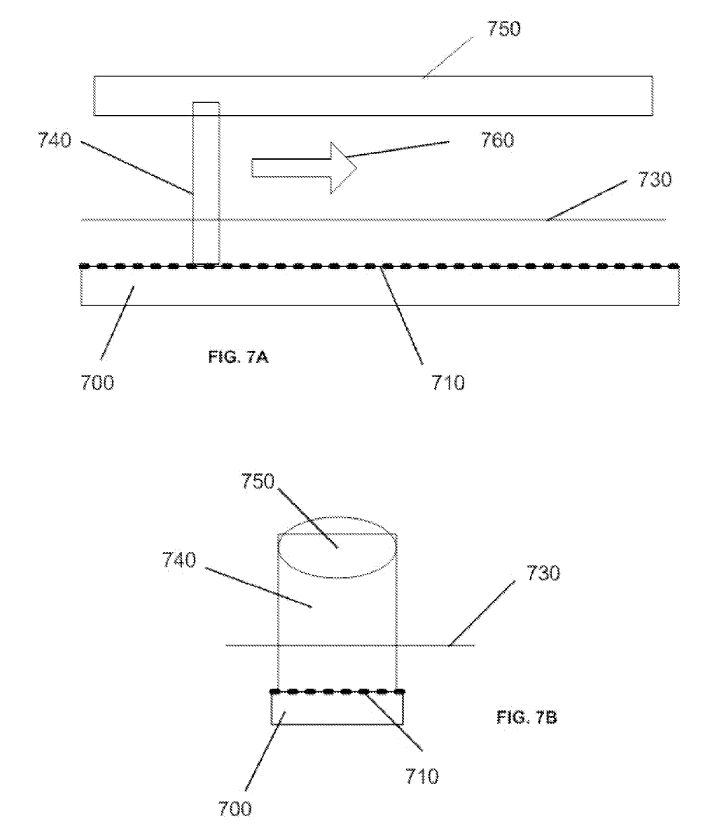

FIG. 6 demonstrates an embodiment where a flat transducer array is used in conjunction with interchangeable lenses. The configurations are the same as those in FIG. 5 with the curved transducer array replaced by a combination of a flat transducer array and a curved lens. In FIG. 6A, flat transducer array 600 has its sound field focused by curved lens 605 with sound field 615 focused on target 610. In FIG. 6B, flat transducer array 630 has its sound field focused by curved lens 635 with sound field 645 focused on target 640. FIG. 6C shows the transducer array 600 with lens 605 of FIG. 6A with sound field 615 focused on target 610 with sound conduction media in place between lens 605 and head 650. The conduction mechanism consists of hemispheric conduction medium 655 and conducting-gel layer 660 providing the physical interface to head 650. These lenses can be bonded to flat transducers or non-permanently affixed. With fixed transducer radii configured to not require beam steering, simpler driving electronics can be used. In some embodiments, a portion of a hemisphere can be used as opposed to a full hemisphere, but in these cases, the power required to achieve a given depth will typically be larger. Different focal depths can be achieved by alterations and different field shapes can be achieved by different array transducer shapes (e.g., curved elongated as opposed to flat linear, square, or hemispheric).

An important reason to use the flat transducer with either a fixed or interchangeable lens is that a simple fixed or variable function generator or equivalent can be used (cost in hundreds to low thousands of dollars) as opposed a beam-steering variable amplitude and phase generator (costs in the tens of thousands of dollars). Representative materials for lens construction are metal or epoxy. In an alternative embodiment, a focusable ultrasound lens can be used (G. A. Brock-Fisher and G. G. Vogel, “Multi-Focus Ultrasound Lens”, U.S. Pat. No. 5,738,098).

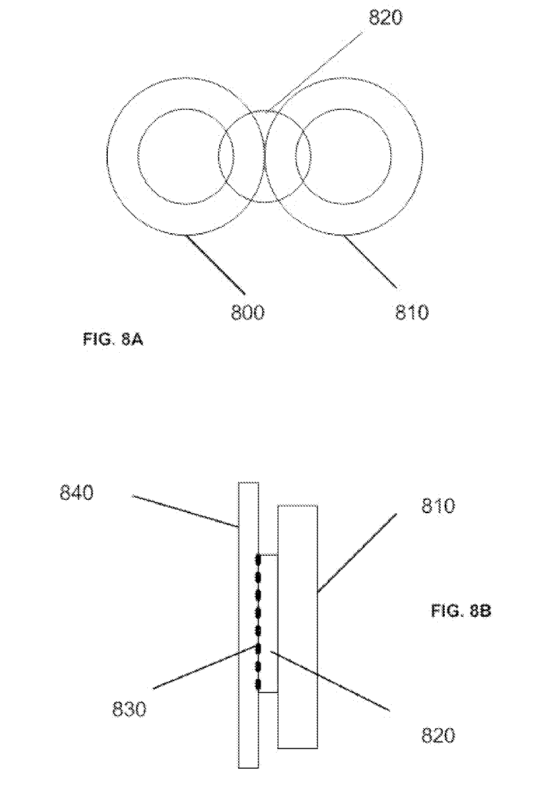

FIG. 7 shows a linear ultrasound phased array with a steered-beam linearly moving field generated by changing the phase/intensity relationships. Beams can also be focused or steered without motion or with non-linear motion. They also can be directed at an angle and not restricted to being aimed perpendicular to the face of the array. FIG. 7A shows a side view and FIG. 7B shows an end view. In FIG. 7A, flat transducer array 700 has its ultrasound conducted by conducting gel layer 710 providing the physical interface to head 730. Sound field 740 moves linearly from left to right as shown by arrow 760 so it moves its focus along target 750. FIG. 7B shows the end view of the configuration looking at the end of flat transducer 700 with conduction of ultrasound to the head 730 provided by conduction layer 710 and sound field 740 focused on target 750. In comparison to FIG. 7A, the sound field 740, which moves, left to right in FIG. 7A moves back into the page in FIG. 7B. In another embodiment, the transducer array is not flat but curved.

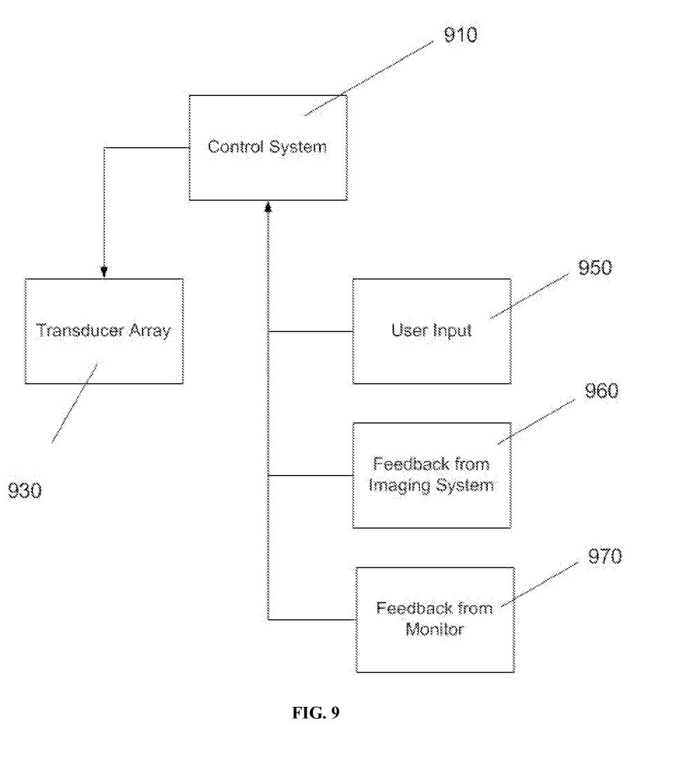

FIG. 8 demonstrates the combination of an ultrasound transducer with a figure-8 Transcranial Magnetic Stimulation (TMS) Coil in both front and side views. FIG. 8A shows the front view of the TMS electromagnet with its component coils 800 and 810 and the face of ultrasonic transducer. The side view of the configuration with the head 840 included is shown in FIG. 8B with the end view of the TMS electromagnet as to side of coil 810, the side of the ultrasound transducer 820. The ultrasound conduction is provided by conductive-gel layer 830 providing the physical interface between ultrasound transducer array 820, and head 840. MRI-compatible ultrasound generators are available (e.g., from Imasonic) so that the presence of the ultrasound transducer will have minimal impact on the magnetic field generated by the TMS electromagnet.

Any shape of array such as those described above may have its sound field steered or focused. The depth of the point where the ultrasound is focused depends on the setting of the phase and amplitude relationships of the elements of the ultrasound transducer array. The same is true for the lateral position of the focus relative to the central axis of the ultrasound transducer array. An example of directing ultrasound is found in Cain and Frizzell (C. A. Cain and L. A. Frizzell, “Apparatus for Generation and Directing Ultrasound,” U.S. Pat. No. 4,549,533). In another embodiment a viewing hole can be placed in an ultrasound transduction to provide an imaging port. Both Imasonic and Keramos-Etalon supply such configurations.

In other embodiments the transducer can be moved back and forth to cover a long target or vibrate in-and-out or in any direction off the central axis to increase the local effects on neural-structure membranes.

FIG. 9 shows a control block diagram. The positioning and emission characteristics of transducer array 930 are controlled by control system 910 with control input from either user by user input 950 and/or from feedback from imaging system 960 (either automatically or display to the user with actual control through user input 950) and/or feedback from a monitor (sound and/or thermal) 970, and/or the patient 980. Control can be provided, as applicable, for direction of the energy emission, intensity, frequency for up-regulation or down-regulation, firing patterns, and phase/intensity relationships for beam steering and focusing on neural targets. In one embodiment control is also provided for a Transcranial Magnetic Stimulation (TMS) coil as integrated with an ultrasound transducer as shown in FIG. 8.

The invention can be applied to a number of conditions including, but not limited to, addiction, Alzheimer's Disease, Anorgasmia, Attention Deficit Hyperactivity Disorder, Huntington's Chorea, Impulse Control Disorder, autism, OCD, Social Anxiety Disorder, Parkinson's Disease, Post-Traumatic Stress Disorder, depression, bipolar disorder, pain, insomnia, spinal cord injuries, neuromuscular disorders, tinnitus, panic disorder, Tourette's Syndrome, amelioration of brain cancers, dystonia, obesity, stuttering, ticks, head trauma, stroke, and epilepsy. In addition it can be applied to cognitive enhancement, hedonic stimulation, enhancement of neural plasticity, improvement in wakefulness, brain mapping, diagnostic applications, and other research functions. In addition to stimulation or depression of individual targets, the invention can be used to globally depress neural activity, which can have benefits, for example, in the early treatment of head trauma or other insults to the brain.

All of the embodiments above, except those explicitly restricted in configuration to hit a single target, are capable of and usually would be used for targeting multiple targets either simultaneously or sequentially. Hitting multiple targets in a neural circuit in a treatment session is an important component of fostering a durable effect through Long-Term Potentiation (LTP) and/or Long-Term Depression (LTD) or enhances acute effects. In addition, this approach can decrease the number of treatment sessions required for a demonstrated effect and to sustain a long-term effect. Follow-up tune-up sessions at one or more later times may be required. In some cases, the neural structures will be targeted bilaterally (e.g., both the right and the left Insula) and in some cases only one will targeted (e.g., the right Insula in the case of addiction).

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

What is claimed is:

1. An ultrasound transducer for neuromodulation of a deep-brain target comprising:
a. an ultrasound-generation array with a curvature matched to the depth of the target, and
b. a shape matched to the shape of the target,
whereby said ultrasound transducer neuromodulates the targeted neural structures producing regulation selected from the group consisting of up-regulation and down-regulation.

2. The device of claim 1, wherein the ultrasound transducer is elongated to match an elongated target.

3. The device of claim 1, wherein the ultrasound transducer is a hemispheric cup shaped to match a point target.

4. The device of claim 1, wherein a plurality of ultrasound transducers are employed to neuromodulate targets selected from the group consisting of multiple targets in a single neural circuit and multiple targets in multiple neural circuits.

5. The device of claim 1, wherein one or plurality of ultrasound transducers are used with one or a plurality of controlled elements selected from the group consisting of direction of the energy emission, intensity, frequency, firing patterns, and phase/intensity relationships for beam steering and focusing on neural targets.

6. An ultrasound transducer for neuromodulation of a deep-brain target comprising:
a. an ultrasound-generation array, and
b. a separate lens shape matched to the depth and shape of the target,
whereby said ultrasound transducer neuromodulates the targeted neural structures producing regulation selected from the group consisting of up-regulation and down-regulation.

7. The system of claim 6, wherein the separate lens used in conjunction with an ultrasound-generating transducer array used in conjunction with the Transcranial Magnetic Stimulation electromagnet has an attachment selected from the group consisting of the bonded to the ultrasound-generating transducer array and not bonded to the ultrasound-generating transducer array.

8. The device of claim 6, wherein the separate lens used in conjunction with the ultrasound generator is interchangeable.

9. The device of claim 6, wherein the separate lens is elongated to match an elongated target

10. The device of claim 6, wherein the separate ultrasound lens is a hemispheric cup shaped to match a point target.

11. An ultrasound transducer for neuromodulation of a deep-brain target comprising:
a. a flat ultrasound-generation array,
b. an ultrasound controller generating varying the phase/intensity relationships to steer and shape the ultrasound beam,
whereby said ultrasound transducer neuromodulates the targeted neural structures producing regulation selected from the group consisting of up-regulation and down-regulation.

12. The device of claim 11, wherein the ultrasound transducer has a curved ultrasound-generation array instead of a flat ultrasound-generation array.

13. The device of claim 11, wherein one or plurality of ultrasound transducers are used with one or a plurality of controlled elements selected from the group consisting of direction of the energy emission, intensity, frequency, firing patterns, and phase/intensity relationships for beam steering and focusing on neural targets.

14. A system for neuromodulation of a deep-brain target comprising:
a. an ultrasound-generation array with a curvature and shaped matched to the depth and shape of the target, and
b. a Transcranial Magnetic Stimulation electromagnet,
whereby said combination ultrasound transducer and Transcranial Magnetic Stimulation electromagnet neuromodulates the targeted neural structures producing regulation selected from the group consisting of up-regulation and down-regulation.

15. The system of claim 14, wherein the separate lens used in conjunction with an ultrasound-generating transducer array used in conjunction with the Transcranial Magnetic Stimulation electromagnet has an attachment selected from the group consisting of the bonded to the ultrasound-generating transducer array and not bonded to the ultrasound-generating transducer array.

16. The system of claim 14, wherein the separate lens used in conjunction with the ultrasound-generating array that is used in conjunction with the Transcranial Magnetic Stimulation electromagnet is interchangeable.

17. The system of claim 14, wherein a plurality of combination ultrasound-generating transducer arrays and Transcranial Magnetic Stimulation electromagnets are employed to neuromodulate targets selected from the group consisting of multiple targets in a neural circuit and multiple targets in multiple neural circuits.

18. The system of claim 14, wherein the combination ultrasound-generating transducer arrays and Transcranial Magnetic Stimulation electromagnets are used with control for the ultrasound-generating transducer arrays of one or a plurality of control elements selected from the group consisting of direction of the energy emission, control of intensity, control of frequency for regulation selected from the group consisting of up-regulation and down-regulation, and control of phase/intensity relationships for beam steering and focusing on neural targets

19. The system of claim 14 wherein the control for the Transcranial Magnetic Stimulation are one or a plurality of control elements selected from the group consisting of intensity, frequency, pulse shape, and timing patterns of the stimulation of the Transcranial Magnetic Stimulation electromagnets.

20. The system of claim 14 wherein the combination of a Transcranial Magnetic Stimulation stimulation means and a coaxial ultrasound transducer array aimed at a neural target increases the neuromodulation of the target to a greater degree than obtainable by either means used alone.
